(19) United States
(12) Patent Application Publication
(10) Pub. No.: US 2019/0358253 A1
(43) Pub. Date: Nov. 28, 2019

(54) COMPOSITION COMPRISING IMMEDIATE RELEASE AND EXTENDED RELEASE CAPECITABINE

(71) Applicant: INTAS PHARMACEUTICALS LTD., Ahmedabad (IN)

(72) Inventors: Kashyap GANDHI, Sanand, Dist.: Ahmedabad (IN); Piyush PATEL, Sanand, Dist.: Ahmedabad (IN); Manish PATEL, Sanand, Dist.: Ahmedabad (IN); Manish CHAUHAN, Sanand, Dist.: Ahmedabad (IN); Ashish SEHGAL, Sanand, Dist.: Ahmedabad (IN)

(73) Assignee: INTAS PHARMACEUTICALS LTD., Ahmedabad (IN)

(21) Appl. No.: 16/483,889

(22) PCT Filed: Feb. 5, 2018
(86) PCT No.: PCT/IB2018/050703

§ 371 Date: Aug. 6, 2019

(30) Foreign Application Priority Data
Feb. 6, 2017 (IN) 201721004194

(51) Int. Cl. A61K 31/7068; A61K 9/24

(52) U.S. Cl. CPC A61K 31/7068; A61K 9/5084; A61K 9/209

(57) ABSTRACT

A pharmaceutical composition of Capecitabine, wherein the composition includes immediate release Capecitabine and extended release Capecitabine. Further disclosed is a process for the preparation of the composition.

### RELATED APPLICATIONS

This application is related to Indian Provisional Application 201721004194 filed 6 Feb. 2017 and is incorporated herein in its entirety.

### FIELD OF THE INVENTION

The present invention relates to a pharmaceutical composition of Capecitabine, wherein the said composition comprises of immediate release Capecitabine and extended release Capecitabine. Further the present invention discloses process for the preparation of the said composition.

### BACKGROUND OF THE INVENTION

Capecitabine is a fluoropyrimidine carbamate having antineoplastic activity. It is an orally administered systemic prodrug of 5′-deoxy-5-fluorouridine (5′-DFUR) which is converted to fluorouracil. Molecular formula of Capecitabine is C₁₅H₂₂FN₃O₆ and the molecular weight is 359.35 and has following chemical structure:

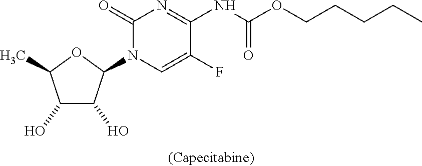 [SMILES: CCCCCOC(=O)Nc1nc(=O)n([C@@H]2O[C@H](C)[C@@H](O)[C@H]2O)cc1F]

U.S. Pat. Nos. 4,966,891 and 5,472,949 discloses Fluorocytidine derivatives and N⁴-(substituted-oxycarbonyl)-5′-deoxy-5-fluorocytidine compounds respectively which cover Capecitabine and methods of using same. Capecitabine is marketed as immediate release tablet comprising 150 or 500 mg Capecitabine for oral administration under trade name XELODA® by Roche. The inactive ingredients in XELODA® include: anhydrous lactose, croscarmellose sodium, hydroxypropyl methylcellulose, microcrystalline cellulose, magnesium stearate and purified water. The peach or light peach film coating contains hydroxypropyl methylcellulose, talc, titanium dioxide, and synthetic yellow and red iron oxides. Capecitabine is indicated for colon cancer, metastatic colorectal cancer and metastatic breast cancer as monotherapy or combination therapy.

Recommended standard starting dose of Capecitabine is 1250 mg/m² administered orally twice daily (morning and evening; equivalent to 2500 mg/m² total daily dose) for 2 weeks followed by a 1-week rest period given as 3-week cycles. In combination with docetaxel, the recommended dose of Capecitabine is 1250 mg/m² twice daily for 2 weeks followed by a 1-week rest period, combined with docetaxel at 75 mg/m² as 1-hour intravenous infusion every 3 weeks. Capecitabine Tablets should be swallowed with water within 30 minutes after the end of a meal.

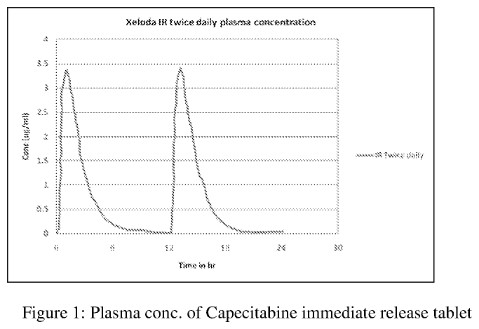

Currently available immediate release (IR) composition of Capecitabine has Tₘₐₓ of approximately 1.5 hours and T_{1/2} of 0.75 hours (FIG. 1). Further the available composition does not maintain constant plasma concentration i.e difficult to achieve steady state concentration.

After administration of currently available IR composition of Capecitabine, the plasma concentration of a Capecitabine reaches below minimum effective concentration after approximately 6 hours, which result in no therapeutic effect between 6 to 12 hours after administration of the dose.

Capecitabine has high therapeutic value for the treatment of cancer. IR tablet of Capecitabine may lead to severe gastrointestinal side effects. Till date, several approaches are reported to overcome the problem associated with IR composition.

WO2013030602 discloses an extended release pharmaceutical composition for oral administration comprising substantially amorphous Capecitabine or an analog thereof and optionally an extended release component, wherein the extended release component is present in an amount of less than 200% w/w, relative to the total weight of the Capecitabine or analog thereof.

WO2006110800 discloses a multiparticulate modified release composition comprising Capecitabine, wherein, following oral delivery, the composition delivers Capecitabine in a pulsatile manner.

US20070122481 discloses a multiparticulate modified release composition comprising a Capecitabine, wherein, following oral delivery, the composition delivers the Capecitabine in a pulsatile manner.

However, still there is need to develop Capecitabine composition which overcomes problems associated with currently available IR tablet, and releases the drug from the composition comprises immediate release Capecitabine and extended release Capecitabine up to 12 hours after administration, which in-turn shall provide and maintain effective plasma concentration of Capecitabine for approximately 12 hours.

### OBJECT OF THE INVENTION

It is therefore object of the invention is to provide a pharmaceutical composition of Capecitabine, wherein the said composition comprises immediate release Capecitabine and extended release Capecitabine, wherein the dissolution of Capecitabine from the said composition is extended up to 12 hours.

Another object of the present invention is to provide a pharmaceutical composition of Capecitabine, wherein the composition comprises immediate release Capecitabine sufficient to provide initial loading dose and remaining amount of Capecitabine as extended release, wherein the dissolution of Capecitabine from the said composition is extended up to 12 hours.

Another object of the present invention is to provide a pharmaceutical composition of Capecitabine, wherein the composition comprises 20% Capecitabine as immediate release and 80% Capecitabine as extended release of the total weight of Capecitabine in the composition, wherein the dissolution of Capecitabine from the said composition is extended up to 12 hours.

Another object of the present invention is to provide an extended release capsule of Capecitabine, wherein the extended release capsule comprises immediate release Capecitabine tablets sufficient to provide initial loading dose and extended release Capecitabine tablets, wherein the dissolution of Capecitabine from the said capsule is extended up to 12 hours.

Another object of the present invention is to provide an extended release capsule of Capecitabine, wherein the extended release capsule comprises 20% Capecitabine as immediate release tablets and 80% Capecitabine as extended release tablets, wherein the amount of Capecitabine is based on the total amount of the Capecitabine in the capsule and dissolution of Capecitabine from the said capsule is extended up to 12 hours.

Another object of the present invention is to provide process for the preparation of an extended release capsule of Capecitabine, wherein the extended release capsule comprises 20% Capecitabine as immediate release tablets and 80% Capecitabine as extended release tablets, wherein the amount of Capecitabine is based on the total amount of the Capecitabine in the capsule and dissolution of Capecitabine from the said capsule is extended up to 12 hours.

Another object of the present invention is to provide bilayer tablet of Capecitabine, wherein the bilayer tablet comprises immediate release Capecitabine layer sufficient to provide initial loading dose and extended release Capecitabine layer, wherein the dissolution of Capecitabine from the said bilayer tablet is extended up to 12 hours.

Another object of the present invention is to provide bilayer tablet of Capecitabine, wherein the bilayer tablet comprises 20% Capecitabine in immediate release layer and 80% Capecitabine in extended release layer, wherein the amount of Capecitabine is based on the total amount of the Capecitabine in the bilayer tablet and dissolution of Capecitabine from the said bilayer tablet is extended up to 12 hours.

Another object of the present invention is to provide process for the preparation of bilayer tablet of Capecitabine, wherein the bilayer tablet comprises 20% Capecitabine in immediate release layer and 80% Capecitabine in extended release layer, wherein the amount of Capecitabine is based on the total amount of the Capecitabine in the bilayer tablet and dissolution of Capecitabine from the said bilayer tablet is extended up to 12 hours.

Another object of the present invention is to provide an extended release capsules comprising multiple units of Capecitabine, wherein the multiple units are in form of mini tablets or pellets or spheres and the multiple units comprises immediate release Capecitabine and extended release Capecitabine, wherein dissolution of Capecitabine from the said multiple units of Capecitabine is extended up to 12 hours.

Another object of the present invention is to provide an extended release capsules comprising multiple units of Capecitabine, wherein the multiple units are in form of mini tablets or pellets or spheres and the extended release capsule comprises 20% Capecitabine as immediate release multiple units and 80% Capecitabine as extended release multiple units and the amount of Capecitabine is based on the total amount of the Capecitabine in the capsule and dissolution of Capecitabine from the said capsule is extended up to 12 hours.

Another object of the present invention is to provide a stable pharmaceutical composition of Capecitabine, wherein the said composition comprises immediate release Capecitabine and extended release Capecitabine, wherein the dissolution of Capecitabine from the said composition is extended up to 12 hours and wherein the total impurity in the said pharmaceutical composition is not more than 2% w/w after subjecting the said pharmaceutical composition to a storage condition of 40° C./75% RH for a period of 2 month.

Another object of the present invention is to provide process for the preparation of an extended release capsules comprising multiple units of Capecitabine, wherein the multiple units are in form of mini tablets or pellets or spheres and the extended release capsule comprises 20% Capecitabine as immediate release multiple units and 80% Capecitabine as extended release multiple units and the amount of Capecitabine is based on the total amount of the Capecitabine in the capsule and dissolution of Capecitabine from the said capsule is extended up to 12 hours.

### SUMMARY OF THE INVENTION

Present invention provides a pharmaceutical composition of Capecitabine, wherein the composition comprises immediate release Capecitabine and extended release Capecitabine. Further the invention provides process for the preparation of the said composition.

### DETAILED DESCRIPTION OF THE INVENTION

Present invention provides a pharmaceutical composition of Capecitabine, wherein the composition comprises immediate release Capecitabine and extended release Capecitabine, wherein the dissolution of Capecitabine from the said composition is extended up to 12 hours.

In another embodiment, the present invention provides a pharmaceutical composition of Capecitabine, wherein the composition comprises immediate release Capecitabine sufficient to provide initial loading dose and remaining amount of Capecitabine as extended release, wherein the dissolution of Capecitabine from the said composition is extended up to 12 hours.

In another embodiment, the present invention provides a pharmaceutical composition of Capecitabine, wherein the composition comprises 20% Capecitabine as immediate release and 80% Capecitabine as extended release of the total weight of Capecitabine in the composition, wherein the dissolution of Capecitabine from the said composition is extended up to 12 hours.

In another embodiment, the present invention provides an extended release capsule of Capecitabine, wherein the extended release capsule comprises immediate release Capecitabine tablets sufficient to provide initial loading dose and extended release Capecitabine tablets, wherein the dissolution of Capecitabine from the said capsule is extended up to 12 hours.

In another embodiment, the present invention provides an extended release capsule of Capecitabine, wherein the extended release capsule comprises 20% Capecitabine as immediate release tablets and 80% Capecitabine as extended release tablets, wherein the amount of Capecitabine is based on the total amount of the Capecitabine in the capsule and dissolution of Capecitabine from the said capsule is extended up to 12 hours.

In another embodiment, the present invention provides process for the preparation of an extended release capsule of Capecitabine, wherein the extended release capsule comprises 20% Capecitabine as immediate release tablets and 80% Capecitabine as extended release tablets, wherein the amount of Capecitabine is based on the total amount of the Capecitabine in the capsule and dissolution of Capecitabine from the said capsule is extended up to 12 hours.

In another embodiment, the present invention provides bilayer tablet of Capecitabine, wherein the bilayer tablet comprises immediate release Capecitabine layer sufficient to provide initial loading dose and extended release Capecitabine layer, wherein the dissolution of Capecitabine from the said bilayer tablet is extended up to 12 hours.

In another embodiment, the present invention provides bilayer tablet of Capecitabine, wherein the bilayer tablet comprises 20% Capecitabine in immediate release layer and 80% Capecitabine in extended release layer, wherein the amount of Capecitabine is based on the total amount of the Capecitabine in the bilayer tablet and dissolution of Capecitabine from the said bilayer tablet is extended up to 12 hours.

In another embodiment, the present invention provides process for the preparation of bilayer tablet of Capecitabine, wherein the bilayer tablet comprises 20% Capecitabine in immediate release layer and 80% Capecitabine in extended release layer, wherein the amount of Capecitabine is based on the total amount of the Capecitabine in the bilayer tablet and dissolution of Capecitabine from the said bilayer tablet is extended up to 12 hours.

In another embodiment, the present invention provides an extended release capsules comprising multiple units of Capecitabine, wherein the multiple units are in form of mini tablets or pellets or spheres and the multiple units comprises immediate release Capecitabine and extended release Capecitabine, wherein dissolution of Capecitabine from the said multiple units of Capecitabine is extended up to 12 hours.

In another embodiment, the present invention provides an extended release capsules comprising multiple units of Capecitabine, wherein the multiple units are in form of mini tablets or pellets or spheres and the extended release capsule comprises 20% Capecitabine as immediate release multiple units and 80% Capecitabine as extended release multiple units and the amount of Capecitabine is based on the total amount of the Capecitabine in the capsule and dissolution of Capecitabine from the said capsule is extended up to 12 hours.

In another embodiment, the present invention provides a process for the preparation of an extended release capsules comprising multiple units of Capecitabine, wherein the multiple units are in form of mini tablets or pellets or spheres and the extended release capsule comprises 20% Capecitabine as immediate release multiple units and 80% Capecitabine as extended release multiple units and the amount of Capecitabine is based on the total amount of the Capecitabine in the capsule and dissolution of Capecitabine from the said capsule is extended up to 12 hours.

For the purpose of this specification, the term “extended release” means release of drug for a longer duration of time i.e. not immediate release.

For the purpose of this specification, the term “immediate release Capecitabine” means a composition or component or layer or unit comprising Capecitabine which releases the Capecitabine form the composition or component or layer or unit immediately i.e. not extended release.

For the purpose of this specification, the term “extended release Capecitabine” means a composition or component or layer or unit comprising Capecitabine which releases the Capecitabine form the composition or component or layer or unit for a longer duration of time i.e. not immediate release.

The pharmaceutical composition according to present invention comprises of suitable excipients. The suitable excipients are added to formulate dosage forms according to the present invention, the suitable excipients may include, but not limited to binder, diluent, lubricant, glidant, and like thereof.

According to present invention, binder may include, but not limited to acacia, carbomer, carboxymethylcellulose, cellulose microcrystalline, copovidone, gelatin, guar gum, hydroxypropyl cellulose, low-substituted hydroxypropyl cellulose, hydroxypropyl methylcellulose, hydroxypropyl methylcellulose acetate succinate, methyl cellulose, ethyl cellulose, polyethylene oxide, povidone, starch, pregelatinized starch, ammonio methacrylate copolymer and the like, or mixtures thereof.

According to present invention, diluent may include, but not limited to lactose anhydrous, lactose monohydrate, spray dried lactose, dicalcium phosphate, calcium phosphate tribasic, calcium carbonate, calcium sulfate, starch, corn starch, potato starch, wheat starch, pregelatinized starch, microcrystalline cellulose, silicified microcrystalline cellulose, cellulose microcrystalline powdered and the like, or mixtures thereof.

According to present invention, lubricant may include, but not limited to calcium stearate, glyceryl behenate, magnesium stearate, mineral oil light, polyethylene glycol, castor oil, sodium stearyl fumarate, starch, stearic acid, talc, hydrogenated vegetable oil, zinc stearate, sodium benzoate and the like, or mixtures thereof.

According to present invention, glidant may include, but not limited to calcium silicate, magnesium silicate, colloidal silicon dioxide, talc and the like, or mixtures thereof.

In another embodiment, the present invention provides a pharmaceutical composition of Capecitabine, wherein the composition comprises immediate release Capecitabine and extended release Capecitabine, wherein the dissolution of Capecitabine from the said composition is extended up to 12 hours and the composition comprises crystalline or amorphous form of Capecitabine.

In another embodiment, the present invention provides a pharmaceutical composition of Capecitabine, wherein the composition comprises immediate release Capecitabine and extended release Capecitabine, wherein extended release is achieved by using modified release matrix material.

According to present invention modified release matrix material may include hydrophilic matrix material or hydrophobic matrix material or mixture thereof. The modified release matrix material may be present in the amount of 10% to 50% by weight of total weight of extended release Capecitabine.

According to present invention modified release matrix material may include, but not limited to hydroxypropyl methylcellulose (HPMC) with different viscosity grade; for example, HPMC K4M, HPMC K100M, HPMC K100LV, hydroxypropylcellulose, hydroxymethylcellulose, hydroxyethylcellulose, hydroxypropyl ethylcellulose, methylcellulose, ethylcellulose, carboxyethylcellulose, carboxymethyl hydroxyethylcellulose, carbomer, sodium carboxymethylcellulose, polyvinylpyrrolidone, hydrophobic polymers, waxes, fats, long-chained fatty acids, fatty alcohols or corresponding esters or ethers or their mixtures, and the like, or mixtures thereof.

In another embodiment, the present invention provides a pharmaceutical composition of Capecitabine, wherein the composition comprises immediate release Capecitabine and extended release Capecitabine, wherein extended release is achieved by using modified release coating onto multiple units of Capecitabine.

According to present invention modified release coating material may include pH-dependent coating material or pH-independent coating material or mixture thereof. The modified release coating material may be present in the amount of 3% to 30% by weight of total weight of extended release Capecitabine.

According to present invention modified release coating material may include, but not limited to alkyl celluloses such as, e.g. ethyl cellulose, or cellulose esters, such as, e.g. cellulose acetate, cellulose acetate phthalate, hydroxylpropyl methyl cellulose phthalate, poly vinyl acetate phthalate, polymethacrylate, copolymer of ethyl acrylate and methyl methacrylate, ammonio methacrylate copolymers, polyacrylic acid and polyacrylate and methacrylate copolymers, shellac, hydroxyethyl cellulose, hydroxypropyl cellulose, hydroxypropyl methylcellulose, polyvinyl pyrrolidone, and the like, or mixtures thereof.

In another embodiment, the present invention provides a stable pharmaceutical composition of Capecitabine, wherein the said composition comprises immediate release Capecitabine and extended release Capecitabine, wherein the dissolution of Capecitabine from the said composition is extended up to 12 hours and wherein the total impurity in the said pharmaceutical composition is not more than 2% w/w after subjecting the said pharmaceutical composition to a storage condition of 40° C./75% RH for a period of 2 month.

### EXAMPLES

The present invention has been described by way of example only, and it is to be recognized that modifications thereto falling within the scope and spirit of appended claims, and which would be obvious to a person skilled in the art based upon the disclosure herein, are also considered to be within the scope of this invention.

### Example 1

Extended release capsule comprising immediate release (IR) tablets of Capecitabine and extended release (ER) tablets of Capecitabine.

a) Composition of extended release and immediate release tablets

| Sr. | | % W/W | |
|---|---|---|---|
| No. | Ingredients | ER tablet | IR tablet |
| 1 | Capecitabine | 60 to 70% | 60 to 70% |
| 2 | Lactose anhydrous | 2 to 8% | 2 to 8% |
| 3 | Microcrystalline cellulose | 4 to 10% | 8 to 25% |
| 4 | Hydroxypropyl methyl cellulose | 1 to 6% | 1 to 6% |
| | 6 cps | | |
| 5 | Purified water | q.s. | q.s. |
| 6 | Hydroxypropyl methyl cellulose | 10 to 40% | — |
| | K 4M CR | | |
| 7 | Croscarmellose sodium | — | 2 to 6% |
| 8 | Magnesium Stearate | 0.50 to 2% | 0.50 to 2% |

b) Filling of predetermined number of immediate release and extended release tablets into suitable capsule.

Process for the Preparation of Extended Release Capsule:

a) Preparation of common granule:
1. Capecitabine, lactose anhydrous, microcrystalline cellulose, hydroxypropyl methyl cellulose 6 cps were sifted through appropriate sieve and mixed properly.
2. Hydroxypropyl methyl cellulose 6 cps was dissolved in purified water and used to granulate the materials of step 1.
3. Wet mass was dried in dryer.
4. Dried granules were passed through appropriate sieve.

b) Preparation of extended release tablets:
5. Hydroxypropyl methyl cellulose K 4M CR was sifted through appropriate sieve and mixed properly with granules obtained in step 4.
6. Magnesium stearate was sifted through appropriate sieve and mixed properly with materials obtained in step 5.
7. Lubricated blend of step 6 was compressed using appropriate tooling to produce extended release tablets.

c) Preparation of immediate release tablets:
8. Microcrystalline cellulose, Croscarmellose sodium were sifted through appropriate sieve and mixed properly with granules obtained in step 4.
9. Magnesium stearate was sifted through appropriate sieve and mixed properly with materials obtained in step 8.
10. Lubricated blend of step 9 was compressed using appropriate tooling to produce immediate release tablets.

d) Preparation of extended release capsule
11. Appropriate number of extended release and immediate release tablets were filled in empty hard gelatin capsule.

### Example 2

Extended release capsule comprising immediate release (IR) tablets of Capecitabine and extended release (ER) tablets of Capecitabine.

a) Composition of extended release and immediate release tablets

| | | 500 mg | | 150 mg | |
|---|---|---|---|---|---|
| Sr. | | ER | IR | ER | IR |
| No. | Ingredients | tablet | tablet | tablet | tablet |

Dry mix

| | | | | | |
|---|---|---|---|---|---|
| 1 | Capecitabine | 50 | 50 | 30 | 30 |
| 2 | Lactose Anhydrous | 4 | 4 | 2.4 | 2.4 |
| 3 | Microcrystalline Cellulose | 5 | 5 | 3 | 3 |
| 4 | Hydroxypropyl Methyl | 1.5 | 1.5 | 0.9 | 0.9 |
| | Cellulose 6 cps | | | | |

Binder solution

| | | | | | |
|---|---|---|---|---|---|
| 5 | Hydroxypropyl Methyl | 1.5 | 1.5 | 0.9 | 0.9 |
| | Cellulose 6 cp | | | | |
| 6 | Purified Water | q.s. | q.s. | q.s. | q.s. |

Blending and Lubrication

| | | | | | |
|---|---|---|---|---|---|
| 7 | Hydroxypropyl Methyl | 12 | — | 7.2 | — |
| | Cellulose K 4M CR | | | | |
| 8 | Microcrystalline cellulose | — | 9 | — | 5.4 |
| 9 | Croscarmellose Sodium | — | 3 | — | 1.8 |
| 10 | Magnesium Stearate | 1 | 1 | 0.6 | 0.6 |
| | Total weight | 75 | 75 | 45 | 45 |

b) Filling of predetermined number of immediate release and extended release tablets into suitable capsule.

| Strength | ER Tablets | IR Tablets | Capsule Shell |
|---|---|---|---|
| 500 mg | 8 Nos. of ER Tablets | 2 Nos. of IR Tablets | EHG Size ′00 |
| 150 mg | 4 Nos. of ER Tablets | 1 No. of IR Tablet | EHG Size ′0el |

Process for the Preparation of Extended Release Capsule:
1. Extended release capsule of the example 2 was prepared by the similar process as of example 1 i.e. from step 1 to step 10.
2. Suitable numbers of tablets were filed into capsule sell. i.e. (a) for preparation of 500 mg capsule, 8 ER tablets and 2 IR tablets were filled into the empty hard gelatin capsule size ‘00 and (b) for preparation of 150 mg capsule, 4 ER tablets and 1 IR tablets were filled into the empty hard gelatin capsule size ‘0e1

### Example 3

Bilayer tablet comprising immediate release (IR) layer of Capecitabine and extended release (ER) layer of Capecitabine.

| | | |
|---|---|---|
| Sr. | | |
| No. | Ingredient | % W/W |

Layer I (ER layer)
Dry mixing

| | | |
|---|---|---|
| 1 | Capecitabine | 60 to 70% |
| 2 | Lactose anhydrous | 2 to 8% |
| 3 | Microcrystalline cellulose | 4 to 8% |
| 4 | Hydroxypropyl methyl cellulose 6 cps | 1 to 3% |

Binder

| | | |
|---|---|---|
| 5 | Hydroxypropyl methyl cellulose 6 cps | 1 to 3% |
| 6 | Purified water | q.s. |

Blending and lubrication

| | | |
|---|---|---|
| 7 | Hydroxypropyl methyl cellulose K 4M CR | 10 to 30% |
| 8 | Magnesium stearate | 0.50 to 2% |

Layer II (IR layer)
Dry mixing

| | | |
|---|---|---|
| 9 | Capecitabine | 30 to 40% |
| 10 | Lactose anhydrous | 38 to 48% |
| 11 | Microcrystalline cellulose | 2 to 6% |
| 12 | Hydroxypropyl methyl cellulose 6 cps | 1 to 3% |

Binder

| | | |
|---|---|---|
| 13 | Hydroxypropyl methyl cellulose 6 cps | 1 to 3% |
| 14 | Purified water | q.s. |

Blending and lubrication

| | | |
|---|---|---|
| 15 | Microcrystalline cellulose | 6 to 12% |
| 16 | Croscarmellose sodium | 1 to 4% |
| 17 | Magnesium stearate | 0.50 to 2% |

Process for the Preparation of Bilayer Tablet:

a) Preparation of common granule:
1. Capecitabine, lactose anhydrous, microcrystalline cellulose, hydroxypropyl methyl cellulose 6 cps were sifted through appropriate sieve and mixed properly.
2. Hydroxypropyl methyl cellulose 6 cps was dissolved in purified water and used to granulate the materials of step 1.
3. Wet mass was dried in dryer.
4. Dried granules were passed through appropriate sieve.

b) Preparation of layer I (ER layer):
5. Hydroxypropyl methyl cellulose K 4M CR was sifted through appropriate sieve and mixed properly with granules obtained in step 4.
6. Magnesium stearate was sifted through appropriate sieve and mixed properly with materials obtained in step 5.
7. Lubricated blend of step 6 was used for the preparation of layer I (ER layer) using bilayer compression machine to prepare bilayer tablet.

c) Preparation of layer II (IR layer):
8. Microcrystalline cellulose, Croscarmellose sodium were sifted through appropriate sieve and mixed properly with granules obtained in step 4.
9. Magnesium stearate was sifted through appropriate sieve and mixed properly with materials obtained in step 8.
10. Lubricated blend of step 9 was used for the preparation of layer II (IR layer) using bilayer compression machine to prepare bilayer tablet.

### Example 4

Bilayer tablet comprising immediate release (IR) layer of Capecitabine and extended release (ER) layer of Capecitabine.

| | | | |
|---|---|---|---|
| Sr. | | 500 mg | 150 mg |
| No. | Ingredient | mg/tab | mg/tab |

Layer I (ER Layer)
Dry mixing

| | | | |
|---|---|---|---|
| 1 | Capecitabine | 400 | 120 |
| 2 | Lactose anhydrous | 32 | 9.6 |
| 3 | Microcrystalline cellulose | 40 | 12 |
| 4 | Hydroxypropyl methyl cellulose 6 cps | 12 | 3.6 |

Binder

| | | | |
|---|---|---|---|
| 5 | Hydroxypropyl methyl cellulose 6 cps | 12 | 3.6 |
| 6 | Purified Water | q.s. | q.s. |

Blending and lubrication

| | | | |
|---|---|---|---|
| 7 | Hydroxypropyl methyl cellulose K 4M CR | 96 | 28.8 |
| 8 | Magnesium stearate | 8 | 2.4 |

| | | |
|---|---|---|
| Total weight of layer I (mg) | 600 | 180 |

Layer II (IR layer)
Dry mixing

| | | | |
|---|---|---|---|
| 9 | Capecitabine | 100 | 30 |
| 10 | Lactose anhydrous | 108 | 32.4 |
| 11 | Microcrystalline cellulose | 10 | 3 |
| 12 | Hydroxypropyl methyl cellulose 6 cps | 3 | 0.9 |

Binder

| | | | |
|---|---|---|---|
| 13 | Hydroxypropyl methyl cellulose 6 cps | 3 | 0.9 |
| 14 | Purified water | q.s. | q.s. |

Blending and lubrication

| | | | |
|---|---|---|---|
| 15 | Microcrystalline cellulose | 18 | 5.4 |
| 16 | Croscarmellose sodium | 6 | 1.8 |
| 17 | Magnesium stearate | 2 | 0.6 |

| | | |
|---|---|---|
| Total weight of layer II (mg) | 250 | 75 |
| Total weight (Layer I + Layer II) (mg) | 850 | 255 |

Process for the Preparation of Bilayer Tablet:

Bilayer tablet of example 4 was prepared by the similar process as of example 3.

### Example 5

Extended release capsule comprising immediate release (IR) tablets of Capecitabine and extended release (ER) tablets of Capecitabine.

a) Composition of extended release and immediate release tablets

| | | 500 mg | | 150 mg | |
|---|---|---|---|---|---|
| Sr. | | ER | IR | ER | IR |
| No. | Ingredients | tablet | tablet | tablet | tablet |

Dry mix

| | | | | | |
|---|---|---|---|---|---|
| 1 | Capecitabine | 200.000 | 100.000 | 120.000 | 30.000 |
| 2 | Lactose Anhydrous | 20.000 | 10.000 | 12.000 | 3.000 |
| 3 | Microcrystalline | 20.000 | 10.000 | 12.000 | 3.000 |
| | Cellulose | | | | |
| 4 | Hydroxypropyl Methyl | 6.000 | 3.000 | 3.600 | 0.900 |
| | Cellulose 6 cps | | | | |

Binder solution

| | | | | | |
|---|---|---|---|---|---|
| 5 | Hydroxypropyl Methyl | 6.000 | 3.000 | 3.600 | 0.900 |
| | Cellulose 6 cp | | | | |
| 6 | Purified Water | q.s. | q.s. | q.s. | q.s. |

Blending and Lubrication

| | | | | | |
|---|---|---|---|---|---|
| 7 | Hydroxypropyl Methyl | 44.000 | — | 26.400 | — |
| | Cellulose K 4M CR | | | | |
| 8 | Microcrystalline | — | 18.000 | — | 5.400 |
| | cellulose | | | | |
| 9 | Croscarmellose Sodium | — | 4.000 | — | 1.200 |
| 10 | Magnesium Stearate | 4.000 | 2.000 | 2.400 | 0.600 |
| | Total weight | 300.000 | 150.000 | 180.000 | 45.000 |

b) Filling of predetermined number of immediate release and extended release tablets into suitable capsule.

| | | | | Average net |
|---|---|---|---|---|
| | | | Capsule | content per |
| Strength | ER Tablets | IR Tablets | Shell | capsule (mg) |
| 500 mg | 2 Nos. of | 1 Nos. of IR | EHG | 300*2 + 150 = 750 |
| | ER Tablets | Tablets | Size ′00 | |
| 150 mg | 1 Nos. of ER | 1 No. of IR | EHG | 180 + 45 = 225 |
| | Tablets | Tablet | Size ′0 | |

EHG = Empty hard gelatin (capsule)

Process for the Preparation of Bilayer Tablet:

Extended release capsule of example 5 was prepared by the similar process as of example 2.

Dissolution Study Results of Example 2:

The dissolution study of the capsules prepared according to examples 2 were carried out in type II dissolution apparatus USP, using 900 ml Phosphate buffer pH 6.8 as dissolution medium at 37° C. and 50 RPM. The obtained dissolution study results are tabulated below:

| | % Drug release | |
|---|---|---|
| | Strength | |
| Time (Hours) | 500 mg | 150 mg |
| 1 | 33 | 37 |
| 2 | 43 | 46 |
| 4 | 58 | 64 |
| 8 | 81 | 90 |
| 12 | 96 | 103 |

Dissolution Study Results of Example 5:

The dissolution study of the capsules prepared according to examples 5 were carried out in type II dissolution apparatus USP, using 900 ml two different dissolution medium i.e. (1) Phosphate buffer pH 6.8 and (2) Acetate buffer pH 4.5 at 37° C. and 50 RPM. The obtained dissolution study results are tabulated below:

| | % Drug release | | | |
|---|---|---|---|---|
| | Strength | | | |
| | 500 mg | | 150 mg | |
| | Dissolution medium | | | |
| | Phosphate | Acetate | Phosphate | Acetate |
| | buffer | buffer | buffer | buffer |
| Time (Hours) | pH 6.8 | pH 4.5 | pH 6.8 | pH 4.5 |
| 1 | 27 | 28 | 29 | 30 |
| 2 | 34 | 36 | 38 | 40 |
| 4 | 47 | 49 | 58 | 56 |
| 8 | 69 | 75 | 78 | 82 |
| 12 | 87 | 93 | 98 | 99 |

Stability Study Results of Example 5:

The stability study of the capsules prepared according to examples 5 was carried out at 40° C./75% RH for 2 months. The stability results obtained are as below.

| | | Test | | | | | |
|---|---|---|---|---|---|---|---|
| | | 500 mg stability study data | | | 150 mg stability study data | | |
| | | | 1M | 2M | | 1M | 2M |
| Sr. No. | Stability condition | Initial | 40° C./75% RH | | Initial | 40° C./75% RH | |
| 1 | Average net content (mg) | 750 | 745.4 | 752.6 | 227.1 | 226.8 | 227.1 |
| 2 | Loss on drying | 2.99% | 2.30% | 2.07% | 1.79% | 2.20% | 2.27% |
| 3 | Assay | 99.60% | 99.10% | 98.90% | 101.50% | 99.30% | 98.90% |
| 4 | Related Substances | | | | | | |
| | A (NMT 1.0%) | 0.04% | 0.14% | 0.25% | 0.05% | 0.15% | 0.29% |
| | B (NMT 1.0%) | 0.04% | 0.03% | 0.04% | 0.04% | 0.03% | 0.04% |
| | C (NMT 0.5%) | BQL | 0.02% | 0.03% | 0.01% | 0.02% | 0.03% |
| | Individual unspecified impurity | ND | ND | BQL | ND | ND | BQL |
| | (NMT 0.1%) | | | | | | |
| | Total impurities (NMT 2.0%) | 0.08% | 0.19% | 0.32% | 0.08% | 0.20% | 0.36% |
| 5 | Dissolution @ 12 hrs in Phosphate buffer pH 6.8/900 ml/type II dissolution apparatus/50 rpm | | | | | | |
| | 1 hrs | 27% | 29% | 27% | 29% | 31% | 31% |
| | 2 hrs | 34% | 37% | 34% | 38% | 41% | 40% |
| | 4 hrs | 47% | 50% | 47% | 58% | 55% | 54% |
| | 8 hrs | 69% | 69% | 69% | 78% | 76% | 75% |
| | 12 hrs | 87% | 87% | 86% | 98% | 95% | 91% |

ND = Not detected,
BQL = Below the quantification limit
Related Substances - A, B, C are as mentioned/defined in official pharmacopoeias i.e. USP 32

Thus, a pharmaceutical composition of Capecitabine, wherein the composition comprises immediate release Capecitabine and extended release Capecitabine can be prepared according to the present invention wherein dissolution of Capecitabine from the said composition is extended up to 12 hours.

We claim:

1. A pharmaceutical composition of Capecitabine, wherein the composition comprises immediate release Capecitabine and extended release Capecitabine, wherein the dissolution of Capecitabine from the said composition is extended up to 12 hours.

2. The pharmaceutical composition according to claim 1, wherein the composition comprises 20% Capecitabine as immediate release and 80% Capecitabine as extended release of the total weight of Capecitabine in the composition.

3. The pharmaceutical composition according to claim 1, wherein the immediate release Capecitabine is sufficient to provide initial loading dose.

4. The pharmaceutical composition according to claim 1, wherein the extended release Capecitabine provides dissolution of Capecitabine from the composition up to 12 hours.

5. The pharmaceutical composition according to claim 1, wherein the immediate release Capecitabine is a composition or component or layer or unit comprising Capecitabine which releases the Capecitabine immediately.

6. The pharmaceutical composition according to claim 1, wherein the extended release Capecitabine is a composition or component or layer or unit comprising Capecitabine which provides dissolution of Capecitabine up to 12 hours.

7. A pharmaceutical composition of Capecitabine, wherein the composition comprises 20% Capecitabine as immediate release and 80% Capecitabine as extended release of the total weight of Capecitabine in the composition and wherein the dissolution of Capecitabine from the said composition is extended up to 12 hours.

8. A pharmaceutical composition of Capecitabine, wherein the total impurity in the said pharmaceutical composition is not more than 2% w/w after subjecting the said pharmaceutical composition to a storage condition of 40° C./75% RH for a period of 2 month.
